(19) United States
(12) Patent Application Publication
(10) Pub. No.: US 2026/0220173 A1
(43) Pub. Date: Jul. 30, 2026

(54) GENERATING A SEQUENCE OF MODEL PROMPTS FOR MACHINE-LEARNING MODELS

(71) Applicant: Formation Bio, Inc., New York, NY (US)

(72) Inventor: John Klingelhofer, Brooklyn, NY (US)

(73) Assignee: Formation Bio, Inc., New York, NY (US)

(21) Appl. No.: 19/460,602

(22) Filed: Jan. 27, 2026

Related U.S. Application Data
(60) Provisional application No. 63/749,932, filed on Jan. 27, 2025.

(51) Int. Cl. G06F 16/3329; G16H 10/20

(52) U.S. Cl. CPC G06F 16/33295; G16H 10/20

(57) ABSTRACT

Disclosed embodiments may provide techniques for generating content using sequences of model prompts to be processed by machine-learning models. A computer-implemented method can include receiving, via a prompt-schema interface, a selection of a machine-learning model from one or more machine-learning models and a sequence of model prompts from a plurality of model prompts. The computer-implemented method can also include processing the sequence of model prompts using the machine-learning model to generate a set of intermediate responses. In some instances, the sequence can be processed by, for each model prompt of the sequence: (i) accessing a previously-generated response; and (ii) processing the model prompt of the sequence and the previously-generated response using the machine-learning model to generate an intermediate response. The computer-implemented method can also include generating a target content based on one or more intermediate responses of the set of intermediate responses.

### CROSS-REFERENCES TO RELATED APPLICATIONS

The present application claims priority from and is a non-provisional of U.S.

Provisional Application No. 63/749,932, entitled “GENERATING A SEQUENCE OF MODEL PROMPTS FOR MACHINE-LEARNING MODELS” filed Jan. 27, 2025, the contents of which are herein incorporated by reference in its entirety for all purposes.

### FIELD

The present disclosure relates generally to generating target content using machine-learning techniques. In one example, the systems and methods described herein may be used to define a sequence of model prompts to be sequentially processed by the machine-learning model to generate the target content.

### SUMMARY

Disclosed embodiments may provide techniques for generating content using sequences of model prompts to be processed by machine-learning models. A computer-implemented method can include providing a prompt-schema interface that identifies one or more machine-learning models for generating content and a plurality of model prompts to be processed by the one or more machine-learning models. In some instances, the prompt-schema interface provides a first set of graphical user-interface elements that represent the one or more machine-learning models, in which the machine-learning model can be selected based on one or more user interactions with the first set of graphical user-interface elements. Additionally or alternatively, the prompt-schema interface can also provide a second set of graphical user-interface elements that represent the sequence of model prompts, in which the sequence of model prompts can be selected based on one or more user interactions with the second set of graphical user-interface elements. The computer-implemented method can also include receiving, via the prompt-schema interface, a selection of a machine-learning model from the one or more machine-learning models and a sequence of model prompts from the plurality of model prompts. In some instances, the machine-learning model is a large-language model (LLM).

The computer-implemented method can also include processing the sequence of model prompts using the machine-learning model to generate a set of intermediate responses. In some instances, the content-generating application sequentially processes the sequence of the model prompts using the machine-learning model.

In some instances, the sequence can be processed by, for each model prompt of the sequence: (i) accessing a previously-generated response, in which the previously-generated response was generated by processing a previous model prompt using the machine-learning model; and (ii) processing the model prompt of the sequence and the previously-generated response using the machine-learning model to generate an intermediate response. In some instances, the previously-generated response corresponds to another intermediate response of the set of intermediate responses, and the previous model prompt corresponds to another model prompt of the sequence. Additionally or alternatively, processing the model prompt can include processing the model prompt, the previously-generated response, and supplemental-input data using the machine-learning model to generate the intermediate response.

In some instances, processing the model prompt includes: (i) determining that the model prompt corresponds to a branching prompt; (ii) processing the branching prompt using the machine-learning model to generate a set of processing threads, in which each processing thread of the set of processing threads is configured to generate a portion of the intermediate response; and (iii) simultaneously processing the set of processing threads in parallel to generate the portions of the intermediate response, in which generating the target content includes aggregating the portions of the intermediate response.

The computer-implemented method can also include generating a target content based on one or more intermediate responses of the set of intermediate responses. The target content can include a data-collection script, a disease report, and/or a patient-recruitment content associated with a clinical trial. In some instances, the target content includes one or more inclusion or exclusion criteria associated with a clinical trial.

In an embodiment, a system comprises one or more processors and memory including instructions that, as a result of being executed by the one or more processors, cause the system to perform the processes described herein. In another embodiment, a non-transitory computer-readable storage medium stores thereon executable instructions that, as a result of being executed by one or more processors of a computer system, cause the computer system to perform the processes described herein.

Various embodiments of the disclosure are discussed in detail below. While specific implementations are discussed, it should be understood that this is done for illustration purposes only. A person skilled in the relevant art will recognize that other components and configurations can be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which can be exhibited by some embodiments and not by others.

The terms used in this specification generally have their ordinary meanings in the art, within the context of the disclosure, and in the specific context where each term is used.

Alternative language and synonyms can be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles can be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

### BRIEF DESCRIPTION OF THE DRAWINGS

Illustrative embodiments are described in detail below with reference to the following figures.

FIG. 1 shows an example computing environment for generating a sequence of model prompts for machine-learning models, in accordance with some embodiments.

FIG. 2 shows an example screenshot of a prompt-schema interface for defining a sequence of model prompts, according to some embodiments.

FIG. 3 shows an example screenshot of a prompt-schema interface that includes two or more model prompts at a particular ordered element of a sequence.

FIG. 4 shows an example set of processing threads generated by processing the branching prompt, according to some embodiments.

FIG. 5 shows an example screenshot of a persona data generated using the sequence of model prompts, according to some embodiments.

FIG. 6 shows an example screenshot of a disease report generated using the sequence of model prompts, according to some embodiments.

FIG. 7 shows an example screenshot of a prescreening form generated using the sequence of model prompts, according to some embodiments.

FIG. 8 shows an illustrative example of a process for generating a sequence of model prompts for machine-learning models, in accordance with some embodiments.

FIG. 9 shows a computing system architecture including various components in electrical communication with each other using a connection in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

The present techniques are directed to generating a chain of prompts to be processed by machine learning models. A prompt-schema interface associated with the prompt schema system allows users to select a particular machine-learning model (e.g., a large-language model). The user may also interact with the prompt-schema interface to create a sequence of prompts for the machine-learning model. The machine-learning model can then process the sequence of prompts to generate a target content. The chaining of prompts allows the machine-learning model to respond to a given prompt by referring to a previous response generated from a previous prompt, thus repeating the process to generate the target content.

The target content generated through the prompt schema can be displayed on a portion of the user interface. The users can interact with and refine the target content. For example, the interaction can include modifying generated text associated with the target content. In some instances, a sidebar can be presented on the user interface, at which the user can initiate a communication session with the large-language model to modify the target content.

In some instances, the present techniques provide a dynamic branching feature.

Dynamic branching can include creating a branching step, in which the branching step identifies a set of input elements (e.g., categories) for being processed in parallel. The subsequent steps from the branching step can be sequentially performed for each input element identified in the branching step. The subsequent steps for the set of input elements can be processed in parallel, which can decrease processing time and increase efficiency in generating the target content.

The present techniques can be used in the context of clinical-trial design. For example, patient personas for clinical trials can be generated from the prompt schema, in which various clinical trial designs can be designed according to the context of the patient persona. Different clinical recruitment, disease reports, and prescreening content can also be generated using the prompt schema. In another example, the sequence of prompts can be configured to generate contextual data, in which the contextual data can be processed by another sequence of prompts to generate the target content.

### I. Techniques for Generating a Sequence of Model Prompts for Machine-Learning Models

### A. Computing Environment

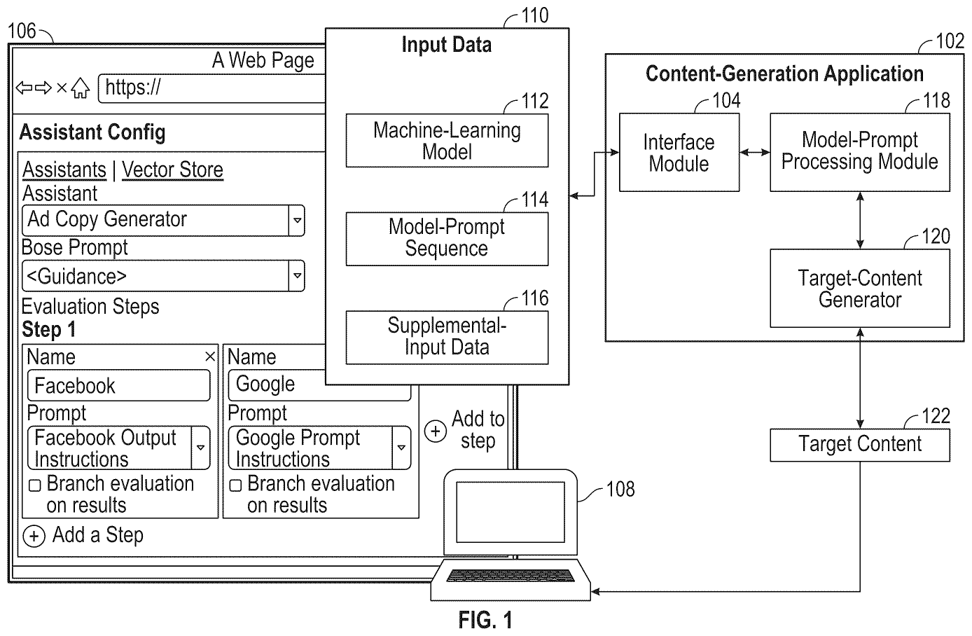

FIG. 1 shows an example computing environment 100 for generating a sequence of model prompts for machine-learning models, in accordance with some embodiments. An interface module 104 of a content-generation application 102 provides a prompt-schema interface 106 that identifies one or more machine-learning models for generating content and a plurality of model prompts to be processed by the one or more machine-learning models. The prompt-schema interface 106 can be configured to facilitate user associated with a user device 108 to select machine-learning models and model prompts to be processed by the selected machine-learning models. In some instances, the model prompts can be specified to be processed in a certain sequence, to allow the machine-learning model to generate the target content.

A model prompt can include one or more queries provided to the machine-learning model to elicit a specific type of response or content. The model prompt can typically include unstructured textual data requesting to generate the target content, as well as specifying the user's intent, context, or constraints for the target content.

To process the model prompts using the machine-learning model (e.g., LLM), a model prompt can be parsed and tokenized. The tokens can be encoded into a high-dimensional vector space using an embedding layer, thus generating an input representation for the model. The machine-learning model can process the input representation through multiple layers of transformers, applying attention mechanisms to capture contextual relationships between the tokens. The machine-learning model then generates a probability distribution over its vocabulary base for each output token based on the learned contextual relationships. By iteratively sampling or selecting tokens from the probability distribution, the machine-learning model can construct a coherent response (e.g., the target content, an intermediate response).

The model prompts can vary in complexity, ranging from simple queries to structured inputs containing examples, instructions, or formatting requirements. Advanced prompting techniques, such as zero-shot, few-shot, and chain-of-thought prompting, can be used to guide the model more effectively for tasks that require reasoning, multi-step logic, or adherence to specific output formats. In some instances, the model prompts can be modified using techniques such as fine-tune prompt wording and structure, to mitigate issues including ambiguity or unintended bias.

In some instances, the model prompt can include one or more configurations that specify different operations to be performed by the content-generation application 102. For example, the content-generation application 102 determines that the model prompt corresponds to a branching prompt, in which the content-generation application 102 can process the branching prompt using the machine-learning models to generate a set of processing threads to be executed in parallel.

The user device 108 can be a client device that includes a desktop computer system, a laptop or notebook computer system, a tablet computer system, a wearable computer system or interface, an interactive kiosk, a mainframe, a mesh of computer systems, a mobile telephone, a personal digital assistant (PDA), or a combination of two or more of these.

A machine-learning model can include a natural-language processing model trained to parse unstructured and structured data associated with the model prompts. Examples of the machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model can include, but are not limited to, genetic algorithms, backpropagation, reinforcement learning, decision trees, linear classification, artificial neural networks, anomaly detection, and such. In yet other examples, the machine-learning model may include regression analysis, dimensionality reduction, metalearning, reinforcement learning, deep learning, and other such algorithms and/or methods. Embodiments for identifying failure indicators using machine-learning techniques are further described in Section II of the present disclosure.

In some instances, the machine-learning model is a transformer model (e.g., a large-language model (LLM)) obtained from a models database. In some instances, the machine-learning model is trained using self-supervised learning based on a large corpus of text data. In addition to training the model, various prompts can be used for prompt engineering of the machine-learning model for generating the qualification indicator. Examples of the machine-learning model can include, but are not limited to, BERT model, Claude LLM, Falcon 40B, Ernie, GPT-3, GPT-3.5, GPT 4, Lamda, and Llama.

In some instances, the prompt-schema interface 106 provides various types of graphical user-interface elements to a user device 108 for selecting machine-learning models and corresponding model prompts. For example, the prompt-schema interface 106 provides a first set of graphical user-interface elements that represent the one or more machine-learning models and a second set of graphical user-interface elements that represent the model prompts. Examples of graphical user-interface elements can include buttons, drop-down menus, checkboxes, radio buttons, text fields, sliders, toggle switches, and any combinations thereof.

The interface module 104 receives, via the prompt-schema interface 106, input data 110, which includes a selection of a machine-learning model 112 from the one or more machine-learning models and a sequence of model prompts 114 from the plurality of model prompts. The sequence of model prompts 114 can define a specific order or series of model prompts to be processed by the selected machine-learning model. Once the sequence 114 is defined, the content-generation application 102 can process the model prompts using the machine-learning model 112, in accordance with the specific order or series defined by the sequence 114. Various types of data structures can be used to specify the sequence 114 of model prompts, including but not limited to arrays, linked lists, stacks, queues, deques, trees (e.g., binary trees, binary search trees), heaps, graphs (with ordered traversal), and hash tables (with ordered keys).

The interface module 104 can receive the input data 110 via a communication network. The network can be any network including an internet, an intranet, an extranet, a cellular network, a Wi-Fi network, a local area network (LAN), a wide area network (WAN), a satellite network, a Bluetooth® network, a virtual private network (VPN), a public switched telephone network, an infrared (IR) network, an internet of things (IoT network) or any other such network or combination of networks. Communications by the client device via the network can be wired connections, wireless connections, or combinations thereof. Communications via the network can be made via a variety of communications protocols including, but not limited to, Transmission Control Protocol/Internet Protocol (TCP/IP), User Datagram Protocol (UDP), protocols in various layers of the Open System Interconnection (OSI) model, File Transfer Protocol (FTP), Universal Plug and Play (UPnP), Network File System (NFS), Server Message Block (SMB), Common Internet File System (CIFS), and other such communications protocols.

In some instances, the user interacts with the first and second sets of graphical user-interface elements to select the machine-learning model 112 and define the sequence 114 of the model prompts. In some instances, the sequence of model prompts 114 can be defined by clicking the corresponding graphical user-interface elements in a specific order, or dragging the graphical user-interface elements to show a particular sequence 114. Other examples of the interactions include clicking, dragging, hovering, double-clicking, swiping, pinching or spreading gestures, tapping with one or more fingers, and combinations thereof.

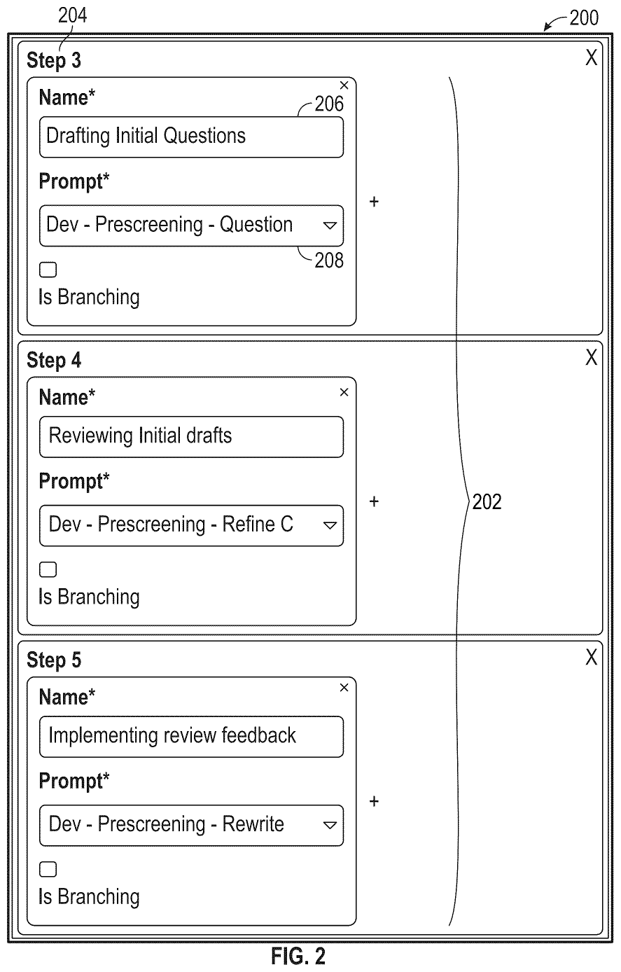

FIG. 2 shows an example screenshot 200 of a prompt-schema interface for defining a sequence of model prompts, according to some embodiments. In FIG. 2, a sequence of model prompts 202 can be defined using the prompt-schema interface. Each model prompt can be associated with a position 204 of the sequence, as shown by “Step 3” element. In some instances, the model prompt can be associated with an identifier 206. For example, the identifier 206 can indicate a description or purpose of the model prompt being provided to the machine-learning model 112. In addition, the model prompt can include the prompt 208, which include unstructured data that specifies instructions to be processed by the machine-learning model 112 to generate the corresponding response. As previously described herein, the user may select the prompt 208 from a plurality of prompts that are pre-loaded and stored in the database.

Additionally or alternatively, the user may input the prompt 208 manually (e.g., using a keyboard).

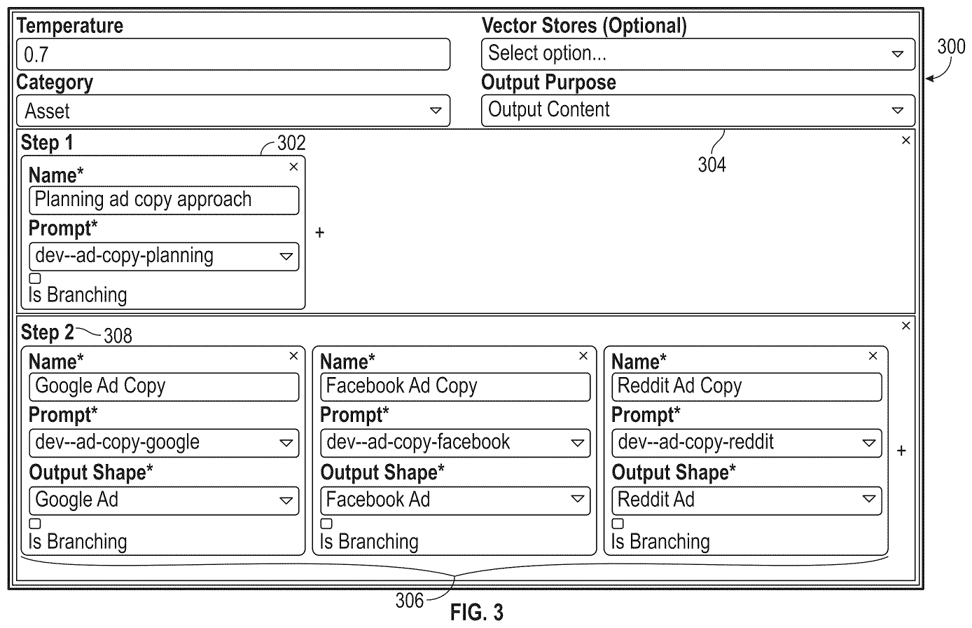

In some instances, each n-th element in the sequence 114 (e.g., first index) can include a model prompt to be processed by the machine-learning model 112. This allows the output generated by the model prompt (e.g., an intermediate response) to be used as additional input for the (n+1)-th element in the sequence 114. Additionally or alternatively, a particular n-th element of the sequence 114 can include two or more model prompts to be processed by the machine-learning model 112, in which the outputs corresponding to the two or more model prompts can be used as additional input for the (n+1)-th element in the sequence 114. FIG. 3 shows an example screenshot 300 of a prompt-schema interface that includes two or more model prompts at a particular position of a sequence. For example, the prompt-schema interface identifies a single model prompt 302 at position 304, at which the response generated by the model prompt 302 can be used with the three model prompts 306 at position 308.

In some instances, the input data 110 can also include supplemental-input data 116 that can include any files or multimodal data (e.g., images, videos, audio data) can be associated with one or more model prompts of the sequence 114. For example, the supplemental-input data 116 can be uploaded and submitted with the sequence of model prompts 114. Examples of the supplemental-input data 116 can include prescreening forms, previous clinical-trial results, institution review board (IRB) protocols and feedback, inclusion and exclusion criteria, and clinical study designs. In some instances, the supplemental-input data 116 can be used as input for a subset of the sequence of model prompts.

A model-prompt processing module 118 of the content-generation application 102 processes the sequence of model prompts 114 using the machine-learning model 112 to generate a set of intermediate responses. In some instances, the content-generation application 102 sequentially processes the sequence 114 of the model prompts using the machine-learning model 112. For example, the content-generation application 102 processes a first model prompt of the sequence 114 to generate a first intermediate response. Turning to the next model prompt of the sequence 114, the content-generation application 102 processes a second model prompt of the sequence 114 and the first intermediate response to generate a second intermediate response. The content-generation application 102 iterates through the above process until an intermediate response for the last model prompt is generated. The set of intermediate responses can then be outputted for further processing.

Stated differently, the content-generation application 102 processes the sequence 114 by, for each model prompt of the sequence 114: (i) accessing a previously-generated response, wherein the previously-generated response was generated by processing a previous model prompt using the machine-learning model 112; and (ii) processing the model prompt of the sequence 114 and the previously-generated response using the machine-learning model 112 to generate an intermediate response. In some instances, the previously-generated response corresponds to another intermediate response of the set of intermediate responses, and the previous model prompt corresponds to another model prompt of the sequence 114.

An illustrative example of training and fine-tuning the machine-learning model 112 is as follows. First, a machine-learning architecture of the machine-learning model 112 can be determined by a training system. The machine-learning model can be generated based on different types of machine-learning architectures. An example architecture used for transformer models can include a transformer model that includes an encoder and a decoder. Another example can include a Bidirectional Encoder Representations from Transformers (BERT), which is configured to understand the context of a word in search queries by considering the words on both its left and right.

In yet another example, a machine-learning architecture can include a Generative Pre-trained Transformer (GPT) that is trained using autoregressive language modeling and masked self-attention techniques. For example, the masked self-attention techniques can include masking future tokens when generating a contextual representation representing a given token, such that the contextual representation is determined only based on past tokens. The autoregressive language modeling techniques can then predict the next token of an output sequence based on the contextual representations of the text tokens.

Other examples of machine-learning architectures can include: (1) a Text-to-Text Transfer Transformer (T5) that converts all natural-language processing tasks into a text-to-text format, unifying various tasks under a single model architecture; and (2) a Vision Transformer (ViT) that extends the transformer architecture to process longer text sequences and image data, respectively, thereby facilitating the corresponding model to be used across different domains.

Once the machine-learning architecture is selected, the machine-learning model 112 can be trained using a training dataset. An illustrative example process of training the transformer model (e.g., a GPT model) is as follows. For the training dataset (e.g., the previous model prompts and corresponding content such as prescreening forms), the masked self-attention process can begin by transforming each word in a given training text sequence into three vectors: the query (Q), key (K), and value (V) vectors. A Q vector can represent what information the token is querying about other tokens, a K vector can represent the token's context used to establish relationships with other tokens, and a V vector can represent the token's actual content/information. In some instances, the Q, K, and V vectors can be obtained by multiplying the input embeddings by learned weight matrices.

An attention score for a particular word can be calculated by taking the dot product of the Q vector of the word with the K vectors of all words in the sequence, thereby producing a score that reflects the relevance of each word pair. The attention scores can be used as weights, which can be applied to the Q, K, V vectors to generate a weighted contextual representation of the particular word. Stated differently, the attention score can be used as a weight to transform the Q, K, V vectors of a given word to generate a weighted, computed representation that can be used to train the corresponding transformer model.

In some instances, a mask can be applied to the self-attention mechanism such that a contextual representation of a given token is determined without weights associated with future tokens. As a result, an attention score of a particular token can be adjusted to disregard information from tokens that have not been processed yet. The attention scores can then be scaled by the square root of the key dimension to stabilize training and passed through a softmax function to convert the attention scores into probabilities, ensuring they sum to one. The transformation can identify the most relevant words while downplaying less important ones. The resulting attention weights can then be used to compute a weighted sum of the V vectors, thus producing a new contextual representation for each token that incorporates contextual information from the entire sequence.

To enhance the model's ability to capture various types of relationships, self-attention mechanisms can use multiple sets of Q, K, and V matrices, also referred to as multi-head attention. Each set, or head, can learn different aspects of the relationships within the input data. The outputs from these heads can be concatenated and linearly transformed to form the final self-attention output. This multi-head approach allows the transformer models to simultaneously consider different features and interactions, enriching its understanding of the input sequence.

The transformer model can then be trained using autoregressive language modeling to predict a subsequent token of a target sequence based on the contextual representations that represent the preceding tokens. For each position in the sequence, the transformer model accesses a contextual representation of the token, which was generated using masked self-attention mechanism. The transformer model can then output a probability distribution over a vocabulary for the subsequent token, conditioned on the sequence of preceding tokens. The subsequent token can then be compared with a corresponding token of the training data to calculate a loss. The loss measures the discrepancy between the predicted token and the actual token, providing a signal for the model to adjust its parameters. The loss can then be used to adjust parameters of the transformer model, including the parameters of the Q, K, V matrices.

Through iterative training iterations, the transformer model learns to minimize this loss across the entire training dataset. This process ensures that the model generates coherent and contextually appropriate sequences by leveraging the learned representations and adjusting its parameters based on the training data.

In some instances, the model-prompt processing module 118 can construct one or more prompts to enhance and increase the accuracy of the target content. In some instances, the model prompt includes a sequence of text tokens in a specific format (e.g., text, XML data, JSON data) and language (e.g., English, Korean).

In some instances, the prompts are machine-generated prompts that are generated by one or more computer systems without user intervention. For example, the one or more model prompts can be constructed using prompt engineering. Prompt engineering can include techniques for designing and implementing prompts within a machine-learning system to generate target responses or actions. In some instances, prompt engineering leverages a combination of linguistic approaches, machine-learning algorithms, and domain knowledge to formulate prompts that elicit specific outputs from a corresponding machine-learning model. The prompt engineering process typically begins with an analysis of a target or a problem domain, followed by the formulation of prompts tailored to achieve the desired results.

As an illustrative example for optimizing prompts, a prompt P can be defined as a sequence of tokens, tailored to elicit specific responses from a machine-learning model. The model employs an objective function O(P, R) to evaluate the quality of generated responses R given the prompt P. The responses R can be generated based on a machine-learning language model LM processing the prompt P (e.g., the function LM(P)). Different types of objective functions can be selected depending on the task and targeted output. For example, an objective function can correspond to a text summarization technique using ROUGE scores. In another example, the objective function can correspond to a translation quality assessment technique using BLEU scores. In some instances, optimization techniques like gradient descent or evolutionary algorithms are used iteratively refine the prompt P to maximize O(P,R), to facilitate the model to consistently produce accurate, relevant, and contextually appropriate outputs (e.g., the model-generated narrative content 422). For example, the optimal prompt P* can be determine based on maximizing the objective function O:

P*=argmax O(P,LM(P)) Equation (1)

Through the iterative refinement process, prompt engineering enhances the corresponding model's performance across various natural language processing tasks, such as generating the ta target content at are contextually relevant to the sequence of model prompts 114.

In some instances, prompt engineering includes a selection of input formats and structures. The input-format selection can include determining the syntactic and semantic characteristics of the prompts that will effectively guide the machine-learning model towards the desired outputs. In some instances, linguistics and computational linguistics can be used to select input formats that are semantically meaningful and contextually relevant. The input-format selection can ensure that the prompts effectively communicate the desired tasks or questions to the machine-learning model. The prompt engineering process can also include an optimization of prompt parameters. The optimization can include fine-tuning various parameters such as prompt length, complexity, and specificity to enhance the machine-learning model's performance on targeted tasks. Different prompt formulations and configurations such as grid search or Bayesian optimization can be implemented to optimize the prompt parameters. Additionally or alternatively, techniques such as zero-shot learning or few-shot learning can be implemented to fine-tune the machine-learning models to generalize from limited prompt examples.

The prompt engineering process can be configured based on an underlying machine-learning model architecture and training data. For example, an appropriate pre-trained machine-learning model architecture (e.g., GPT, BERT, or Transformer) that aligns with the task requirements and available computational resources can be identified for a given task. In some instances, the machine-learning model can be fine-tuned on task-specific data to further improve probability of outputting target responses. Various types of training datasets can be used to train and fine-tune the machine-learning model, so as to enable the machine-learning model to understand and generate responses to prompts accurately.

In some instances, an iterative process of designing, testing, and optimizing prompts is implemented based on feedback from initial model outputs. This iterative approach allows for continuous improvement and refinement of the prompt engineering process, ultimately leading to better-performing machine-learning models. Additionally or alternatively, ongoing monitoring and evaluation of model performance can be used to identify any errors or biases introduced by the prompts and prompt engineering process, in which the feedback data can be generated based on the evaluation. The feedback data can be used to further adjust the parameters of the machine-learning models, such that the machine-learning models can be updated to improve accuracy in generating the target responses.

Once training is performed, the model-prompt processing module 118 can apply the trained and fine-tuned machine-learning model 112 to the sequence 114 to generate the intermediate responses. To begin the deployment process, the model-prompt processing module 118 can tokenize the model prompts into a sequence of text tokens. For example, the multimodal data can be tokenized to provide the following sequence: [“You”, “are”, “an”, “assistant”, “tasked”, . . . ]. In some instances, the machine-learning model uses Byte Pair Encoding (BPE) techniques to further split a single token (e.g., “in”, “sufficient”).

The model-prompt processing module 118 can assign each token with a particular index value in the vocabulary (e.g., “assistant”=E[5]). Then, the model-prompt processing module 118 can convert each token into a vector representation (e.g., an embedding) based on a pre-trained embedding matrix. For example, for a vocabulary size V and embedding dimension dᵢ, the embedding matrix E is of size V×d, in which the vector eᵢ can be generated for the text token tᵢ based on using the index value a looking of a corresponding row of embedding matrix E.

E:eᵢ=E[tᵢ] Equation (2)

The model-prompt processing module 118 can then process the sequence of embeddings (e₁, e₂, e₃, . . . eₙ) that represent the sequence of tokens by adding positional encodings to account for the order of tokens. In some instances, positional encodings are vectors added to each token embedding to inject information about the position of tokens in the sequence. A matrix X can be formed that includes the sequence of position-encoded vectors. In some instances, the model-prompt processing module 118 can also encode the intermediate responses generated for the previous model prompts into another sequence of embeddings, such that the model-prompt processing module 118 can generate the subsequent intermediate responses based on the information generated from the previous model prompts of the sequence.

For the matrix X, the model-prompt processing module 118 can then determine a contextual representation for each position-encoded vector of the matrix X. In particular, for each position-encoded vector, the model-prompt processing module 118 can generate a set of Q, K, V vectors for the position-encoded vector. As described herein, a Q vector can represent what information the token is querying about other tokens, a K vector can represent the token's context used to establish relationships with other tokens, and a V vector can represent the token's actual content/information.

In some instances, to enhance the model's ability to capture various types of relationships, the position-encoded vector can be represented by multiple sets of Q, K, and V matrices (i.e., multi-head attention). Each set of Q, K, V vectors, or head, can learn different aspects of the relationships within the input data. The outputs from these heads can be concatenated and linearly transformed to form the final self-attention output. This multi-head approach allows the transformer models to simultaneously consider different features and interactions, enriching its understanding of the input sequence.

An attention score can be calculated for the set of Q, K, V vectors as follows:

Attention(Q,K,V)=softmax((QK^{T})/√(dₖ))V —Equation (3)

The (QK^{T})/√(dₖ) can be used to compute the raw attention scores, in which dₖ is the dimensionality of the key vectors. Then, the softmax function is applied to the raw attention score to normalize it into a probability distribution. The model-prompt processing module 118 can apply the attention score to a V vector of the corresponding set of Q, K, V vectors, such that the weighted Q, K, V vectors can be used as the contextual representation of the position-encoded vector of matrix X In the instances in which multi-head attention is used, the multiple sets of weighted Q, K, V vectors can be concatenated and linearly transformed using a weight matrix W^{O} to generate the contextual representation of the position-encoded vector. The above process can be iterated through other position-encoded vectors of matrix X to generate a set of contextual representations associated with the multimodal data.

The model-prompt processing module 118 can then apply the machine-learning model 112 to the set of contextual representations to generate the intermediate responses corresponding to each ordered element of the sequence of model prompts 114. In particular, the machine-learning model can process the set of contextual representations to predict each token of the output, in which the outputted tokens can correspond to the intermediate responses.

In some instances, the intermediate responses can be augmented using data accessed from a retrieval-augmented generation (RAG) system. The RAG system can be configured to optimize the outputs (e.g., the machine-generated responses) of the machine-learning model 112 by referencing an external knowledge base that is outside of the training data used for training the machine-learning model 112. In some instances, the outputs are associated with the prompt associated with a user.

To generate the abovementioned outputs, the RAG system can access a knowledge base stored in database server. The knowledge base can include a repository that stores information associated with a product, service, domain, or a topic, which can be used to supplement the intermediate responses generated by machine-learning model. In some instances, the knowledge base includes domain-specific information, which can be associated with a particular domain.

Examples of domains can include data sources associated with clinical trial protocol development, regulatory guidelines, ethical considerations, statistical design, clinical trial phases, operational aspects, data management, adverse event reporting, therapeutic areas, patient-centric approaches, emerging trends, external data sources, and cost and feasibility.

In some instances, when processing the branching prompt, the content-generation application 102 processes the branching prompt using the machine-learning model 112 to a set of processing threads, in which each processing thread of the set of processing threads is configured to generate a portion of the intermediate response. The content-generation application 102 then simultaneously processes the set of processing threads in parallel to generate the portions of the intermediate response. The portions of the intermediate response can be aggregated, in which the aggregated portions can be used to generate the target content.

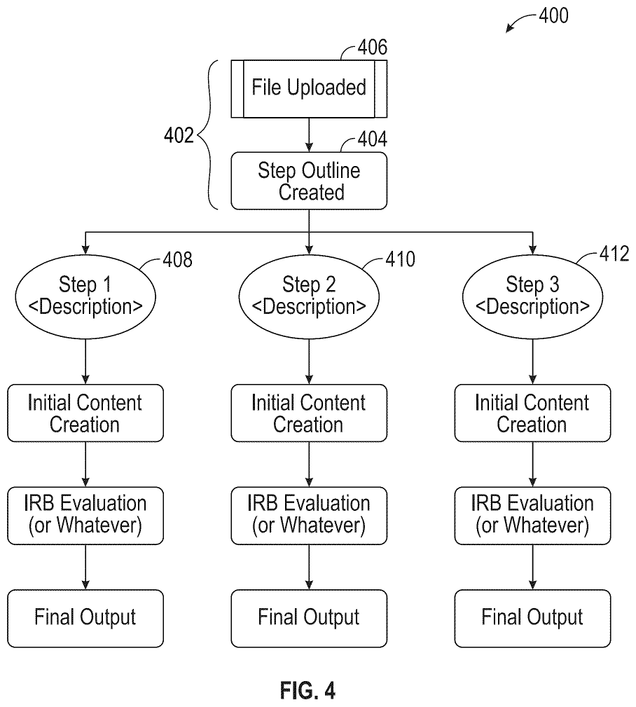

FIG. 4 illustrates an example set of processing threads 400 generated by processing the branching prompt, according to some embodiments. As shown in FIG. 4, a branching prompt 402 can include unstructured data 404 that identifies an outline of steps to be executed by each processing thread. The model-prompt processing module 118 can process the branching prompt 402 by generating a set of processing threads 408, 410, and 412, in which the machine-learning model can simultaneously process the set of processing threads 408, 401, and 412 in parallel, in accordance with the steps outlined in the unstructured data 404 of the branching prompt 402.

Additionally or alternatively, the model-prompt processing module 118 can process the branching prompt 402 with one or more files 406 that may include any relevant information associated with the unstructured data 404.

The number of processing threads can be determined based on the information (e.g., the outline of steps) specified by the branching prompt 402. For example, the number of processing threads can correspond to the number of responses the model is instructed to produce (e.g. 10 processing threads for generating 10 titles for sections in a prescreening form). In another example, the number of processing threads can correspond to the number of communication channels for which the target content is to be generated (e.g., 2 processing threads for generating content for a social media platform and an email platform). The number of processing threads can range from 1, 2, 3, 4, 5, 10, 15, 20, 25, 50, 75, 100, 500, 1000, or more than 1000 processing threads.

Additionally or alternatively, the intermediate responses (and consequently the target content) can be customized based on the supplemental-input data 116 associated with one or more model prompts of the sequence. In particular, the content-generation application 102 can process the model prompt, the previously-generated response, and the supplemental-input data 116 using the machine-learning model 112 to generate the intermediate response.

A target-content generator 120 of the content-generation application 102 generates a target content 122 based on one or more intermediate responses of the set of intermediate responses. In some instances, the target content 122 is generated by aggregating the one or more intermediate responses of the set of intermediate responses. The target content 122 can be generated by selecting and processing the intermediate response of the last model prompt of the sequence 114. Additionally or alternatively, the target content 122 can be generated by processing the set of intermediate responses using another machine-learning model (e.g., a natural language model).

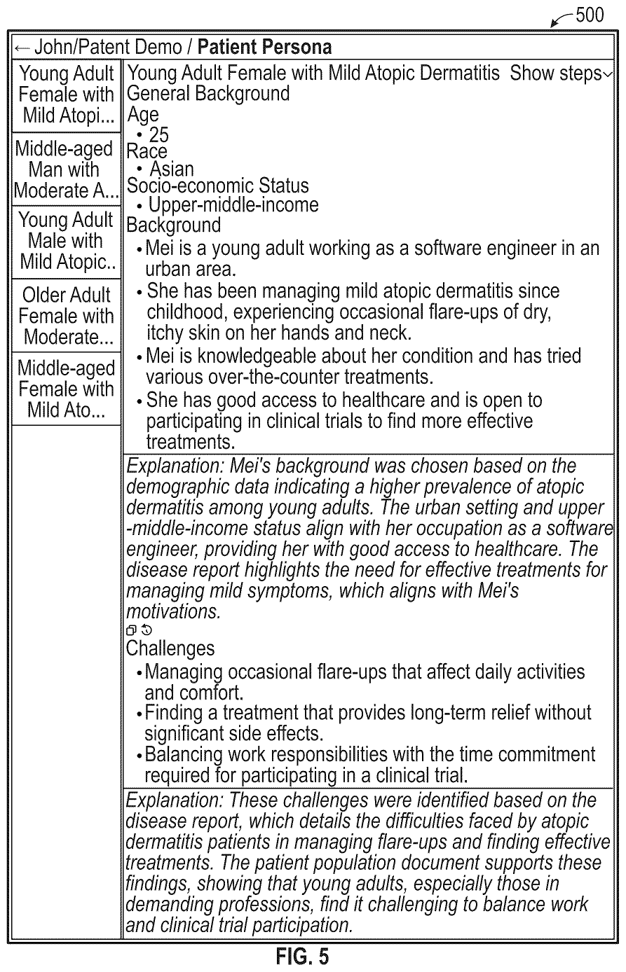

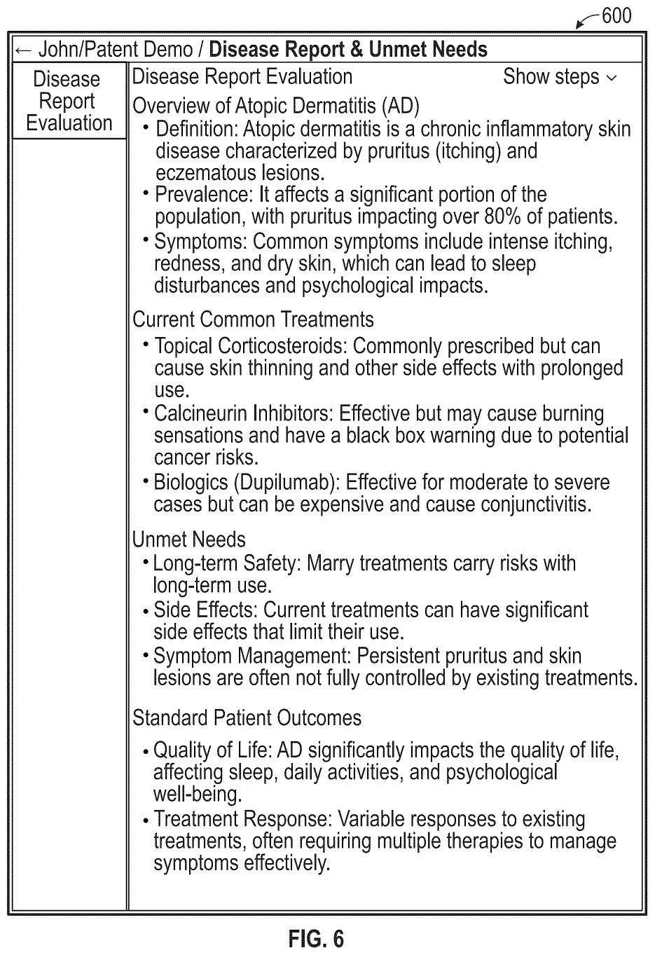

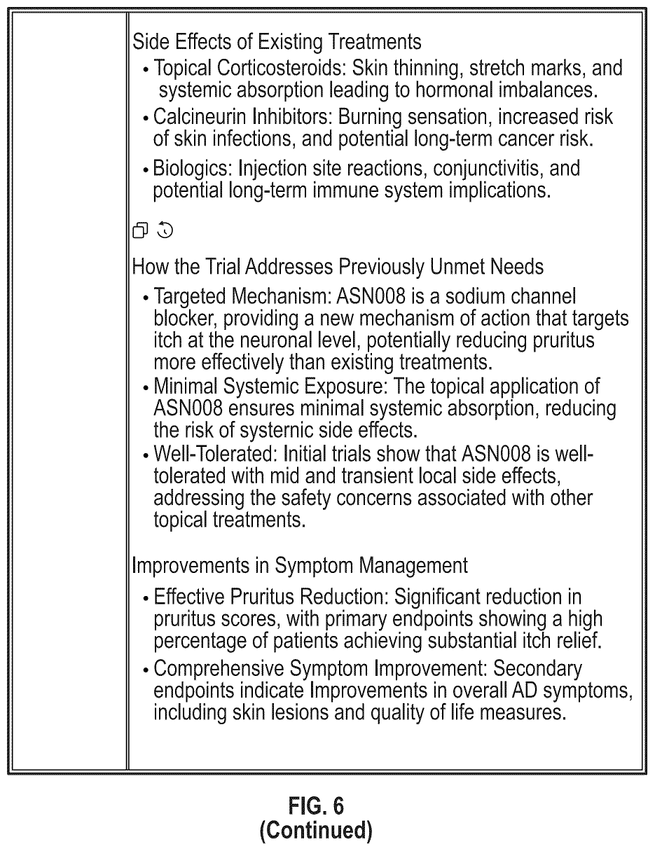

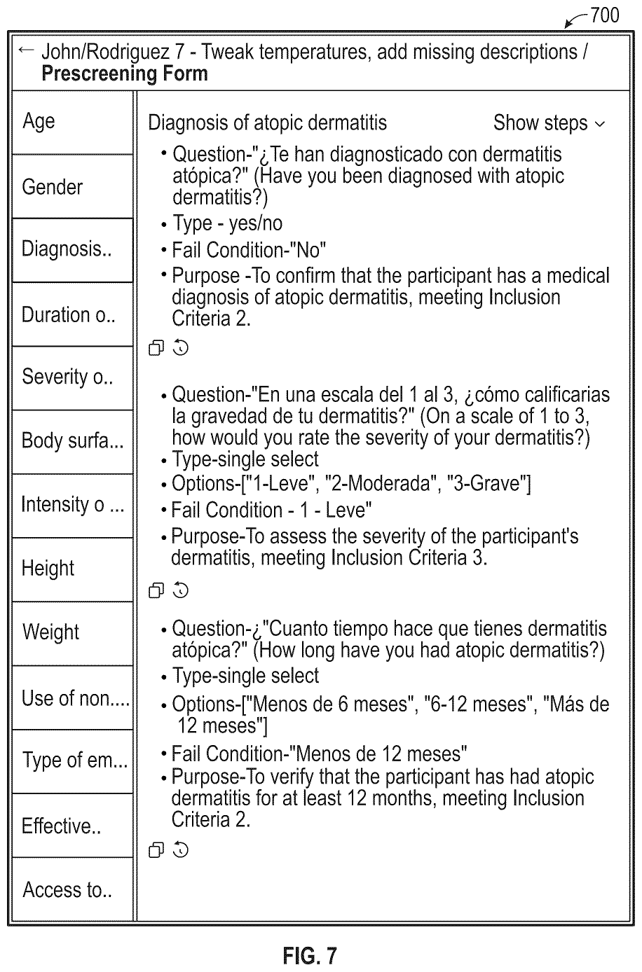

The target content 122 can include a data-collection script, a persona data, a disease report, and/or a prescreening form associated with a clinical trial. In some instances, the target content 122 includes one or more inclusion or exclusion criteria associated with a clinical trial. FIG. 5 shows an example screenshot 500 of a persona data generated using the sequence of model prompts, according to some embodiments. As shown in FIG. 5, the persona data can be generated to identify one or more characteristics (e.g., young adult female with mild atopic dermatitis, age 25) of a subject, which can be used for designing clinical trials and other relevant documents. FIG. 6 shows an example screenshot 600 of a disease report generated using the sequence of model prompts, according to some embodiments. The disease report identifies various aspects of atopic dermatitis in detail, including current common treatments and unmet needs. FIG. 7 shows an example screenshot 700 of a prescreening form generated using the sequence of model prompts, according to some embodiments. The prescreening form in FIG. 7 provides a set of questions for determining diagnosis of atopic dermatitis.

The target content 122 can be outputted, such as displaying the target content 122 on at least a portion of the prompt-schema interface 106, or another graphical-user interface of the user device 108. In some instances, the target content 122 can be transmitted to another device through a communication network. Additionally or alternatively, the prompt-schema interface can initiate a separate communication session with a user (e.g., a chat window displayed on the interface), in which another machine-learning model (e.g., another LLM) can communicate with the user about the target content 122 (e.g., feedback regarding certain types of questions of the prescreening form).

### B. Methods

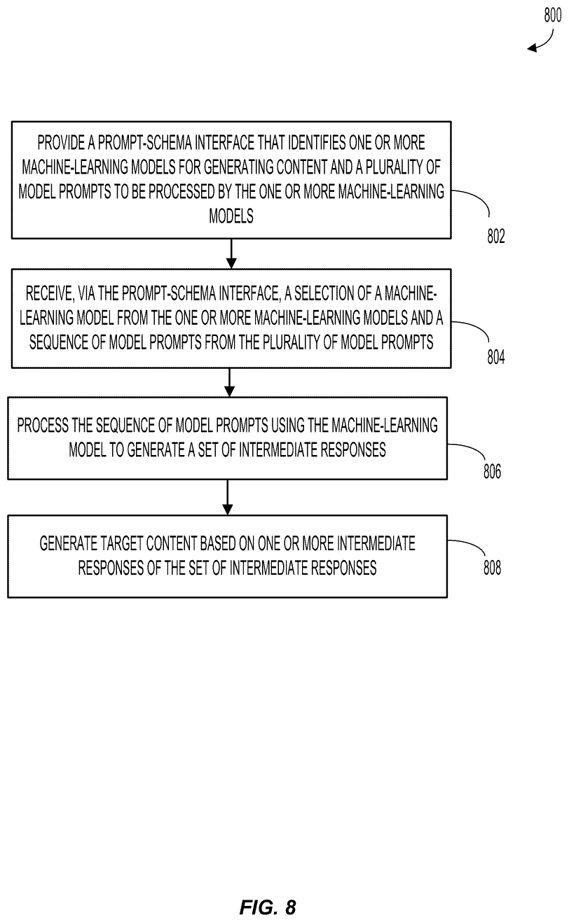

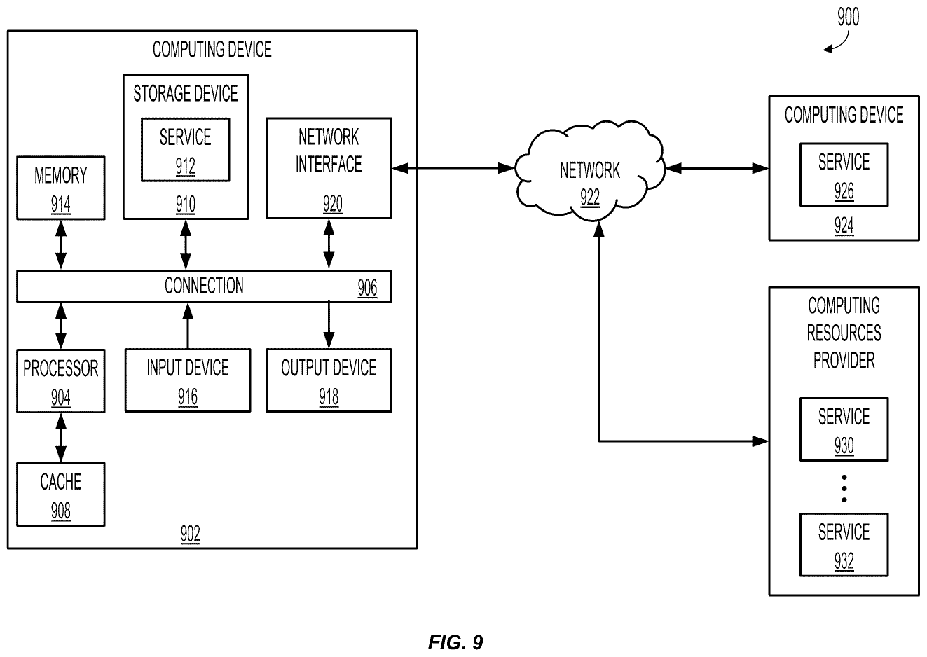

FIG. 8 shows an illustrative example of a process 800 for generating a sequence of model prompts for machine-learning models, in accordance with some embodiments. For illustrative purposes, the process 800 is described with reference to the components illustrated in FIG. 1, though other implementations are possible. For example, the program code for the content-generating application of FIG. 1, is executed by one or more processing devices to cause a server system (e.g., the computing device 902 of FIG. 9) to perform one or more operations described herein.

At step 802, the content-generating application provides a prompt-schema interface that identifies one or more machine-learning models for generating content and a plurality of model prompts to be processed by the one or more machine-learning models. The prompt-schema interface can be configured to facilitate a user device to select machine-learning models and model prompts to be processed by the selected machine-learning model. In some instances, the model prompts can be specified to be processed in a certain sequence, to allow the machine-learning model to generate the target content.

A model prompt can include one or more queries provided to the machine-learning model to elicit a specific type of response or content. The model prompt can typically include unstructured textual data requesting to generate the target content, as well as specifying the user's intent, context, or constraints for the target content.

To process the model prompts using the machine-learning model (e.g., LLM), a model prompt can be parsed and tokenized. The tokens can be encoded into a high-dimensional vector space using an embedding layer, thus generating an input representation for the model. The machine-learning model can process the input representation through multiple layers of transformers, applying attention mechanisms to capture contextual relationships between the tokens. The machine-learning model then generates a probability distribution over its vocabulary base for each output token based on the learned contextual relationships. By iteratively sampling or selecting tokens from the probability distribution, the machine-learning model can construct a coherent response (e.g., the target content, an intermediate response).

The model prompts can vary in complexity, ranging from simple queries to structured inputs containing examples, instructions, or formatting requirements. Advanced prompting techniques, such as zero-shot, few-shot, and chain-of-thought prompting, can be used to guide the model more effectively for tasks that require reasoning, multi-step logic, or adherence to specific output formats. In some instances, the model prompts can be modified using techniques such as fine-tune prompt wording and structure, to mitigate issues including ambiguity or unintended bias.

In some instances, the model prompt can include one or more configurations that specify different operations to be performed by the content-generating application. For example, the content-generating application determines that the model prompt corresponds to a branching prompt, in which the content-generating application can process the branching prompt using the machine-learning models to generate a set of processing threads to be executed in parallel.

The user device can be a client device that includes a desktop computer system, a laptop or notebook computer system, a tablet computer system, a wearable computer system or interface, an interactive kiosk, a mainframe, a mesh of computer systems, a mobile telephone, a personal digital assistant (PDA), or a combination of two or more of these.

A machine-learning model can include a natural-language processing model trained to parse unstructured and structured data associated with the model prompts. Examples of the machine-learning model can include algorithms such as k-means clustering algorithms, fuzzy c-means (FCM) algorithms, expectation-maximization (EM) algorithms, hierarchical clustering algorithms, and density-based spatial clustering of applications with noise (DBSCAN) algorithms, in which the algorithms can be trained using unsupervised learning. Other examples of the machine-learning model can include, but are not limited to, genetic algorithms, backpropagation, reinforcement learning, decision trees, linear classification, artificial neural networks, anomaly detection, and such. In yet other examples, the machine-learning model may include regression analysis, dimensionality reduction, metalearning, reinforcement learning, deep learning, and other such algorithms and/or methods. Embodiments for identifying failure indicators using machine-learning techniques are further described in Section II of the present disclosure.

In some instances, the machine-learning model is a transformer model (e.g., a large-language model (LLM)) obtained from a models database. In some instances, the machine-learning model is trained using self-supervised learning based on a large corpus of text data. In addition to training the model, various prompts can be used for prompt engineering of the machine-learning model for generating the qualification indicator. Examples of the machine-learning model can include, but are not limited to, BERT model, Claude LLM, Falcon 40B, Ernie, GPT-3, GPT-3.5, GPT 4, Lamda, and Llama.

In some instances, the prompt-schema interface provides various types of graphical user-interface elements to a user device for selecting machine-learning models and corresponding model prompts. For example, the prompt-schema interface provides a first set of graphical user-interface elements that represent the one or more machine-learning models and a second set of graphical user-interface elements that represent the model prompts. Examples of graphical user-interface elements can include buttons, drop-down menus, checkboxes, radio buttons, text fields, sliders, toggle switches, and any combinations thereof.

At step 804, the content-generating application receives, via the prompt-schema interface, a selection of a machine-learning model from the one or more machine-learning models and a sequence of model prompts from the plurality of model prompts. The sequence of model prompts can define a specific order or series of model prompts to be processed by the selected machine-learning model. Once the sequence is defined, the content-generating application can process the model prompts using the machine-learning model, in accordance with the specific order or series defined by the sequence.

In some instances, the user interacts with the first and second sets of graphical user-interface elements to select the machine-learning model and define the sequence of the model prompts. For example, the sequence of model prompts can be defined by clicking the corresponding graphical user-interface elements in a specific order, or dragging the graphical user-interface elements to show a particular sequence. Other examples of the interactions include clicking, dragging, hovering, double-clicking, swiping, pinching or spreading gestures, tapping with one or more fingers, and combinations thereof.

At step 806, the content-generating application processes the sequence of model prompts using the machine-learning model to generate a set of intermediate responses. In some instances, the content-generating application sequentially processes the sequence of the model prompts using the machine-learning model. For example, the content-generating application processes a first model prompt of the sequence to generate a first intermediate response. Turning to the next model prompt of the sequence, the content-generating application processes a second model prompt of the sequence and the first intermediate response to generate a second intermediate response. The content-generating application iterates through the above process until an intermediate response for the last model prompt is generated. The set of intermediate responses can then be outputted for further processing.

Stated differently, the content-generating application processes the sequence by, for each model prompt of the sequence: (i) accessing a previously-generated response, in which the previously-generated response was generated by processing a previous model prompt using the machine-learning model; and (ii) processing the model prompt of the sequence and the previously-generated response using the machine-learning model to generate an intermediate response. In some instances, the previously-generated response corresponds to another intermediate response of the set of intermediate responses, and the previous model prompt corresponds to another model prompt of the sequence.

When processing the branching prompt, the content-generating application processes the branching prompt using the machine-learning model to generate a set of processing threads, in which each processing thread of the set of processing threads is configured to generate a portion of the intermediate response. The content-generating application then simultaneously processes the set of processing threads in parallel to generate the portions of the intermediate response. The portions of the intermediate response can be aggregated, in which the aggregated portions can be used to generate the target content.

Additionally or alternatively, the intermediate responses (and consequently the target content) can be customized based on supplemental-input data associated with the sequence of model prompts. For example, the content-generating application accesses the supplemental-input data from a retrieval-augmented generation (RAG) system. The content-generating application can then process the model prompt, the previously-generated response, and the supplemental-input data using the machine-learning model to generate the intermediate response.

At step 808, the content-generating application generates a target content based on one or more intermediate responses of the set of intermediate responses. In some instances, the target content is generated by aggregating the one or more intermediate responses of the set of intermediate responses. The target content can be generated by selecting and processing the intermediate response of the last model prompt of the sequence. Additionally or alternatively, the target content can be generated by processing the set of intermediate responses using another machine-learning model (e.g., a natural language model).

The target content can include a data-collection script, a persona data, a disease report, and/or a patient-recruitment content associated with a clinical trial. In some instances, the target content includes one or more inclusion or exclusion criteria associated with a clinical trial. The target content can be outputted, such as displaying the target content on at least a portion of the prompt-schema interface, or another graphical-user interface of the user device. In some instances, the target content can be transmitted to another device through a communication network. Process 800 terminates thereafter.

### II. Example Systems

FIG. 9 illustrates a computing system architecture 900, including various components in electrical communication with each other, in accordance with some embodiments. The example computing system architecture 900 illustrated in FIG. 9 includes a computing device 902, which has various components in electrical communication with each other using a connection 906, such as a bus, in accordance with some implementations. The example computing system architecture 900 includes a processing unit 904 that is in electrical communication with various system components, using the connection 906, and including the system memory 914. In some embodiments, the system memory 914 includes read-only memory (ROM), random-access memory (RAM), and other such memory technologies including, but not limited to, those described herein. In some embodiments, the example computing system architecture 900 includes a cache 908 of high-speed memory connected directly with, in close proximity to, or integrated as part of the processor 904. The system architecture 900 can copy data from the memory 914 and/or the storage device 910 to the cache 908 for quick access by the processor 904. In this way, the cache 908 can provide a performance boost that decreases or eliminates processor delays in the processor 904 due to waiting for data. Using modules, methods and services such as those described herein, the processor 904 can be configured to perform various actions. In some embodiments, the cache 908 may include multiple types of cache including, for example, level one (L1) and level two (L2) cache. The memory 914 may be referred to herein as system memory or computer system memory. The memory 914 may include, at various times, elements of an operating system, one or more applications, data associated with the operating system or the one or more applications, or other such data associated with the computing device 902.

Other system memory 914 can be available for use as well. The memory 914 can include multiple different types of memory with different performance characteristics. The processor 904 can include any general purpose processor and one or more hardware or software services, such as service 912 stored in storage device 910, configured to control the processor 904 as well as a special-purpose processor where software instructions are incorporated into the actual processor design. The processor 904 can be a completely self-contained computing system, containing multiple cores or processors, connectors (e.g., buses), memory, memory controllers, caches, etc. In some embodiments, such a self-contained computing system with multiple cores is symmetric. In some embodiments, such a self-contained computing system with multiple cores is asymmetric. In some embodiments, the processor 904 can be a microprocessor, a microcontroller, a digital signal processor (“DSP”), or a combination of these and/or other types of processors. In some embodiments, the processor 904 can include multiple elements such as a core, one or more registers, and one or more processing units such as an arithmetic logic unit (ALU), a floating point unit (FPU), a graphics processing unit (GPU), a physics processing unit (PPU), a digital system processing (DSP) unit, or combinations of these and/or other such processing units.

To enable user interaction with the computing system architecture 900, an input device 916 can represent any number of input mechanisms, such as a microphone for speech, a touch-sensitive screen for gesture or graphical input, keyboard, mouse, motion input, pen, and other such input devices. An output device 918 can also be one or more of a number of output mechanisms known to those of skill in the art including, but not limited to, monitors, speakers, printers, haptic devices, and other such output devices. In some instances, multimodal systems can enable a user to provide multiple types of input to communicate with the computing system architecture 900. In some embodiments, the input device 916 and/or the output device 918 can be coupled to the computing device 902 using a remote connection device such as, for example, a communication interface such as the network interface 920 described herein. In such embodiments, the communication interface can govern and manage the input and output received from the attached input device 916 and/or output device 918. As may be contemplated, there is no restriction on operating on any particular hardware arrangement and accordingly the basic features here may easily be substituted for other hardware, software, or firmware arrangements as they are developed.

In some embodiments, the storage device 910 can be described as non-volatile storage or non-volatile memory. Such non-volatile memory or non-volatile storage can be a hard disk or other types of computer readable media which can store data that are accessible by a computer, such as magnetic cassettes, flash memory cards, solid state memory devices, digital versatile disks, cartridges, RAM, ROM, and hybrids thereof.

As described above, the storage device 910 can include hardware and/or software services such as service 912 that can control or configure the processor 904 to perform one or more functions including, but not limited to, the methods, processes, functions, systems, and services described herein in various embodiments. In some embodiments, the hardware or software services can be implemented as modules. As illustrated in example computing system architecture 900, the storage device 910 can be connected to other parts of the computing device 902 using the system connection 906. In some embodiments, a hardware service or hardware module such as service 912, that performs a function can include a software component stored in a non-transitory computer-readable medium that, in connection with the necessary hardware components, such as the processor 904, connection 906, cache 908, storage device 910, memory 914, input device 916, output device 918, and so forth, can carry out the functions such as those described herein.

The disclosed systems and service of a content-generating application (e.g., the content-generating application 102 described herein at least in connection with FIG. 1) can be performed using a computing system such as the example computing system illustrated in FIG. 9, using one or more components of the example computing system architecture 900. An example computing system can include a processor (e.g., a central processing unit), memory, non-volatile memory, and an interface device. The memory may store data and/or and one or more code sets, software, scripts, etc. The components of the computer system can be coupled together via a bus or through some other known or convenient device.

In some embodiments, the processor can be configured to carry out some or all of methods and systems described herein by, for example, executing code using a processor such as processor 904 wherein the code is stored in memory such as memory 914 as described herein. One or more of a user device, a provider server or system, a database system, or other such devices, services, or systems may include some or all of the components of the computing system such as the example computing system illustrated in FIG. 9, using one or more components of the example computing system architecture 900 illustrated herein. As may be contemplated, variations on such systems can be considered as within the scope of the present disclosure.

This disclosure contemplates the computer system taking any suitable physical form. As example and not by way of limitation, the computer system can be an embedded computer system, a system-on-chip (SOC), a single-board computer system (SBC) (such as, for example, a computer-on-module (COM) or system-on-module (SOM)), a desktop computer system, a laptop or notebook computer system, a tablet computer system, a wearable computer system or interface, an interactive kiosk, a mainframe, a mesh of computer systems, a mobile telephone, a personal digital assistant (PDA), a server, or a combination of two or more of these. Where appropriate, the computer system may include one or more computer systems; be unitary or distributed; span multiple locations; span multiple machines; and/or reside in a cloud computing system which may include one or more cloud components in one or more networks as described herein in association with the computing resources provider 928. Where appropriate, one or more computer systems may perform without substantial spatial or temporal limitation one or more steps of one or more methods described or illustrated herein. As an example and not by way of limitation, one or more computer systems may perform in real time or in batch mode one or more steps of one or more methods described or illustrated herein. One or more computer systems may perform at different times or at different locations one or more steps of one or more methods described or illustrated herein, where appropriate.

The processor 904 can be a conventional microprocessor such as an Intel® microprocessor, an AMD® microprocessor, a Motorola® microprocessor, or other such microprocessors. One of skill in the relevant art will recognize that the terms “machine-readable (storage) medium” or “computer-readable (storage) medium” include any type of device that is accessible by the processor.

The memory 914 can be coupled to the processor 904 by, for example, a connector such as connector 906, or a bus. As used herein, a connector or bus such as connector 906 is a communications system that transfers data between components within the computing device 902 and may, in some embodiments, be used to transfer data between computing devices. The connector 906 can be a data bus, a memory bus, a system bus, or other such data transfer mechanism. Examples of such connectors include, but are not limited to, an industry standard architecture (ISA″ bus, an extended ISA (EISA) bus, a parallel AT attachment (PATA″ bus (e.g., an integrated drive electronics (IDE) or an extended IDE (EIDE) bus), or the various types of parallel component interconnect (PCI) buses (e.g., PCI, PCIe, PCI-104, etc.).

The memory 914 can include RAM including, but not limited to, dynamic RAM (DRAM), static RAM (SRAM), synchronous dynamic RAM (SDRAM), non-volatile random access memory (NVRAM), and other types of RAM. The DRAM may include error-correcting code (EEC). The memory can also include ROM including, but not limited to, programmable ROM (PROM), erasable and programmable ROM (EPROM), electronically erasable and programmable ROM (EEPROM), Flash Memory, masked ROM (MROM), and other types or ROM. The memory 914 can also include magnetic or optical data storage media including read-only (e.g., CD ROM and DVD ROM) or otherwise (e.g., CD or DVD). The memory can be local, remote, or distributed.

As described above, the connector 906 (or bus) can also couple the processor 904 to the storage device 910, which may include non-volatile memory or storage and which may also include a drive unit. In some embodiments, the non-volatile memory or storage is a magnetic floppy or hard disk, a magnetic-optical disk, an optical disk, a ROM (e.g., a CD-ROM, DVD-ROM, EPROM, or EEPROM), a magnetic or optical card, or another form of storage for data.

Some of this data may be written, by a direct memory access process, into memory during execution of software in a computer system. The non-volatile memory or storage can be local, remote, or distributed. In some embodiments, the non-volatile memory or storage is optional. As may be contemplated, a computing system can be created with all applicable data available in memory. A typical computer system will usually include at least one processor, memory, and a device (e.g., a bus) coupling the memory to the processor.

Software and/or data associated with software can be stored in the non-volatile memory and/or the drive unit. In some embodiments (e.g., for large programs) it may not be possible to store the entire program and/or data in the memory at any one time. In such embodiments, the program and/or data can be moved in and out of memory from, for example, an additional storage device such as storage device 910. Nevertheless, it should be understood that for software to run, if necessary, it is moved to a computer readable location appropriate for processing, and for illustrative purposes, that location is referred to as the memory herein. Even when software is moved to the memory for execution, the processor can make use of hardware registers to store values associated with the software, and local cache that, ideally, serves to speed up execution. As used herein, a software program is assumed to be stored at any known or convenient location (from non-volatile storage to hardware registers), when the software program is referred to as “implemented in a computer-readable medium.” A processor is considered to be “configured to execute a program” when at least one value associated with the program is stored in a register readable by the processor.

The connection 906 can also couple the processor 904 to a network interface device such as the network interface 920. The interface can include one or more of a modem or other such network interfaces including, but not limited to those described herein. It will be appreciated that the network interface 920 may be considered to be part of the computing device 902 or may be separate from the computing device 902. The network interface 920 can include one or more of an analog modem, Integrated Services Digital Network (ISDN) modem, cable modem, token ring interface, satellite transmission interface, or other interfaces for coupling a computer system to other computer systems. In some embodiments, the network interface 920 can include one or more input and/or output (I/O) devices. The I/O devices can include, by way of example but not limitation, input devices such as input device 916 and/or output devices such as output device 918. For example, the network interface 920 may include a keyboard, a mouse, a printer, a scanner, a display device, and other such components. Other examples of input devices and output devices are described herein. In some embodiments, a communication interface device can be implemented as a complete and separate computing device.

In operation, the computer system can be controlled by operating system software that includes a file management system, such as a disk operating system. One example of operating system software with associated file management system software is the family of Windows® operating systems and their associated file management systems. Another example of operating system software with its associated file management system software is the Linux™ operating system and its associated file management system including, but not limited to, the various types and implementations of the Linux® operating system and their associated file management systems. The file management system can be stored in the non-volatile memory and/or drive unit and can cause the processor to execute the various acts required by the operating system to input and output data and to store data in the memory, including storing files on the non-volatile memory and/or drive unit. As may be contemplated, other types of operating systems such as, for example, MacOS®, other types of UNIX® operating systems (e.g., BSD™ and descendants, Xenix™, SunOS™, HP-UX®, etc.), mobile operating systems (e.g., iOS® and variants, Chrome®, Ubuntu Touch®, watchOS®, Windows 10 Mobile®, the Blackberry® OS, etc.), and real-time operating systems (e.g., VxWorks®, QNX®, eCos®, RTLinux®, etc.) may be considered as within the scope of the present disclosure. As may be contemplated, the names of operating systems, mobile operating systems, real-time operating systems, languages, and devices, listed herein may be registered trademarks, service marks, or designs of various associated entities.

In some embodiments, the computing device 902 can be connected to one or more additional computing devices such as computing device 924 via a network 922 using a connection such as the network interface 920. In such embodiments, the computing device 924 may execute one or more services 926 to perform one or more functions under the control of, or on behalf of, programs and/or services operating on computing device 902. In some embodiments, a computing device such as computing device 924 may include one or more of the types of components as described in connection with computing device 902 including, but not limited to, a processor such as processor 904, a connection such as connection 906, a cache such as cache 908, a storage device such as storage device 910, memory such as memory 914, an input device such as input device 916, and an output device such as output device 918. In such embodiments, the computing device 924 can carry out the functions such as those described herein in connection with computing device 902. In some embodiments, the computing device 902 can be connected to a plurality of computing devices such as computing device 924, each of which may also be connected to a plurality of computing devices such as computing device 924. Such an embodiment may be referred to herein as a distributed computing environment.

The network 922 can be any network including an internet, an intranet, an extranet, a cellular network, a Wi-Fi network, a local area network (LAN), a wide area network (WAN), a satellite network, a Bluetooth® network, a virtual private network (VPN), a public switched telephone network, an infrared (IR) network, an internet of things (IoT network) or any other such network or combination of networks. Communications via the network 922 can be wired connections, wireless connections, or combinations thereof. Communications via the network 922 can be made via a variety of communications protocols including, but not limited to, Transmission Control Protocol/Internet Protocol (TCP/IP), User Datagram Protocol (UDP), protocols in various layers of the Open System Interconnection (OSI) model, File Transfer Protocol (FTP), Universal Plug and Play (UPnP), Network File System (NFS), Server Message Block (SMB), Common Internet File System (CIFS), and other such communications protocols.

Communications over the network 922, within the computing device 902, within the computing device 924, or within the computing resources provider 928 can include information, which also may be referred to herein as content. The information may include text, graphics, audio, video, haptics, and/or any other information that can be provided to a user of the computing device such as the computing device 902. In some embodiments, the information can be delivered using a transfer protocol such as Hypertext Markup Language (HTML), Extensible Markup Language (XML), JavaScript®, Cascading Style Sheets (CSS), JavaScript® Object Notation (JSON), and other such protocols and/or structured languages. The information may first be processed by the computing device 902 and presented to a user of the computing device 902 using forms that are perceptible via sight, sound, smell, taste, touch, or other such mechanisms. In some embodiments, communications over the network 922 can be received and/or processed by a computing device configured as a server. Such communications can be sent and received using PUP: Hypertext Preprocessor (“PHP”), Python™, Ruby, Perl® and variants, Java®, HTML, XML, or another such server-side processing language.

In some embodiments, the computing device 902 and/or the computing device 924 can be connected to a computing resources provider 928 via the network 922 using a network interface such as those described herein (e.g. network interface 920). In such embodiments, one or more systems (e.g., service 930 and service 932) hosted within the computing resources provider 928 (also referred to herein as within “a computing resources provider environment”) may execute one or more services to perform one or more functions under the control of, or on behalf of, programs and/or services operating on computing device 902 and/or computing device 924.

Systems such as service 930 and service 932 may include one or more computing devices such as those described herein to execute computer code to perform the one or more functions under the control of, or on behalf of, programs and/or services operating on computing device 902 and/or computing device 924.

For example, the computing resources provider 928 may provide a service, operating on service 930 to store data for the computing device 902 when, for example, the amount of data that the computing device 902 exceeds the capacity of storage device 910. In another example, the computing resources provider 928 may provide a service to first instantiate a virtual machine (VM) on service 932, use that VM to access the data stored on service 932, perform one or more operations on that data, and provide a result of those one or more operations to the computing device 902. Such operations (e.g., data storage and VM instantiation) may be referred to herein as operating “in the cloud,” “within a cloud computing environment,” or “within a hosted virtual machine environment,” and the computing resources provider 928 may also be referred to herein as “the cloud.” Examples of such computing resources providers include, but are not limited to Amazon® Web Services (AWS®), Microsoft's Azure®, IBM Cloud®, Google Cloud®, Oracle Cloud® etc.

Services provided by a computing resources provider 928 include, but are not limited to, data analytics, data storage, archival storage, big data storage, virtual computing (including various scalable VM architectures), blockchain services, containers (e.g., application encapsulation), database services, development environments (including sandbox development environments), e-commerce solutions, game services, media and content management services, security services, server-less hosting, virtual reality (VR) systems, and augmented reality (AR) systems. Various techniques to facilitate such services include, but are not be limited to, virtual machines, virtual storage, database services, system schedulers (e.g., hypervisors), resource management systems, various types of short-term, mid-term, long-term, and archival storage devices, etc.

As may be contemplated, the systems such as service 930 and service 932 may implement versions of various services (e.g., the service 912 or the service 926) on behalf of, or under the control of, computing device 902 and/or computing device 924. Such implemented versions of various services may involve one or more virtualization techniques so that, for example, it may appear to a user of computing device 902 that the service 912 is executing on the computing device 902 when the service is executing on, for example, service 930. As may also be contemplated, the various services operating within the computing resources provider 928 environment may be distributed among various systems within the environment as well as partially distributed onto computing device 924 and/or computing device 902.

Client devices, user devices, computer resources provider devices, network devices, and other devices can be computing systems that include one or more integrated circuits, input devices, output devices, data storage devices, and/or network interfaces, among other things. The integrated circuits can include, for example, one or more processors, volatile memory, and/or non-volatile memory, among other things such as those described herein. The input devices can include, for example, a keyboard, a mouse, a key pad, a touch interface, a microphone, a camera, and/or other types of input devices including, but not limited to, those described herein. The output devices can include, for example, a display screen, a speaker, a haptic feedback system, a printer, and/or other types of output devices including, but not limited to, those described herein. A data storage device, such as a hard drive or flash memory, can enable the computing device to temporarily or permanently store data. A network interface, such as a wireless or wired interface, can enable the computing device to communicate with a network. Examples of computing devices (e.g., the computing device 902) include, but is not limited to, desktop computers, laptop computers, server computers, hand-held computers, tablets, smart phones, personal digital assistants, digital home assistants, wearable devices, smart devices, and combinations of these and/or other such computing devices as well as machines and apparatuses in which a computing device has been incorporated and/or virtually implemented.

The techniques described herein may also be implemented in electronic hardware, computer software, firmware, or any combination thereof. Such techniques may be implemented in any of a variety of devices such as general purpose computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as that described herein. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices (e.g., a combination of a DSP and a microprocessor), a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for implementing a suspended database update system.

As used herein, the term “machine-readable media” and equivalent terms “machine-readable storage media,” “computer-readable media,” and “computer-readable storage media” refer to media that includes, but is not limited to, portable or non-portable storage devices, optical storage devices, removable or non-removable storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), solid state drives (SSD), flash memory, memory or memory devices.

A machine-readable medium or machine-readable storage medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like. Further examples of machine-readable storage media, machine-readable media, or computer-readable (storage) media include but are not limited to recordable type media such as volatile and non-volatile memory devices, floppy and other removable disks, hard disk drives, optical disks (e.g., CDs, DVDs, etc.), among others, and transmission type media such as digital and analog communication links.

As may be contemplated, while examples herein may illustrate or refer to a machine-readable medium or machine-readable storage medium as a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding, or carrying a set of instructions for execution by the system and that cause the system to perform any one or more of the methodologies or modules of disclosed herein.

Some portions of the detailed description herein may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “generating” or the like, refer to the action and processes of a computer system, or similar electronic computing device, that manipulates and transforms data represented as physical (electronic) quantities within registers and memories of the computer system into other data similarly represented as physical quantities within the computer system memories or registers or other such information storage, transmission or display devices.

It is also noted that individual implementations may be described as a process which is depicted as a flowchart, a flow diagram, a data flow diagram, a structure diagram, or a block diagram (e.g., the example process 800 of FIG. 8). Although a flowchart, a flow diagram, a data flow diagram, a structure diagram, or a block diagram may describe the operations as a sequential process, many of the operations can be performed in parallel or concurrently. In addition, the order of the operations may be re-arranged. A process illustrated in a figure is terminated when its operations are completed, but could have additional steps not included in the figure. A process may correspond to a method, a function, a procedure, a subroutine, a subprogram, etc. When a process corresponds to a function, its termination can correspond to a return of the function to the calling function or the main function.

In some embodiments, one or more implementations of an algorithm such as those described herein may be implemented using a machine learning or artificial intelligence algorithm. Such a machine learning or artificial intelligence algorithm may be trained using supervised, unsupervised, reinforcement, or other such training techniques. For example, a set of data may be analyzed using one of a variety of machine learning algorithms to identify correlations between different elements of the set of data without supervision and feedback (e.g., an unsupervised training technique). A machine learning data analysis algorithm may also be trained using sample or live data to identify potential correlations. Such algorithms may include k-means clustering algorithms, fuzzy c-means (FCM) algorithms, expectation-maximization (EM) algorithms, hierarchical clustering algorithms, density-based spatial clustering of applications with noise (DBSCAN) algorithms, and the like. Other examples of machine learning or artificial intelligence algorithms include, but are not limited to, genetic algorithms, backpropagation, reinforcement learning, decision trees, liner classification, artificial neural networks, anomaly detection, and such. More generally, machine learning or artificial intelligence methods may include regression analysis, dimensionality reduction, metalearning, reinforcement learning, deep learning, and other such algorithms and/or methods. As may be contemplated, the terms “machine learning” and “artificial intelligence” are frequently used interchangeably due to the degree of overlap between these fields and many of the disclosed techniques and algorithms have similar approaches.

As an example of a supervised training technique, a set of data can be selected for training of the machine learning model to facilitate identification of correlations between members of the set of data. The machine learning model may be evaluated to determine, based on the sample inputs supplied to the machine learning model, whether the machine learning model is producing accurate correlations between members of the set of data. Based on this evaluation, the machine learning model may be modified to increase the likelihood of the machine learning model identifying the desired correlations. The machine learning model may further be dynamically trained by soliciting feedback from users of a system as to the efficacy of correlations provided by the machine learning algorithm or artificial intelligence algorithm (i.e., the supervision). The machine learning algorithm or artificial intelligence may use this feedback to improve the algorithm for generating correlations (e.g., the feedback may be used to further train the machine learning algorithm or artificial intelligence to provide more accurate correlations).

The various examples of flowcharts, flow diagrams, data flow diagrams, structure diagrams, or block diagrams discussed herein may further be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable storage medium (e.g., a medium for storing program code or code segments) such as those described herein. A processor(s), implemented in an integrated circuit, may perform the necessary tasks.

The various illustrative logical blocks, modules, circuits, and algorithm steps described in connection with the implementations disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

It should be noted, however, that the algorithms and displays presented herein are not inherently related to any particular computer or other apparatus. Various general purpose systems may be used with programs in accordance with the teachings herein, or it may prove convenient to construct more specialized apparatus to perform the methods of some examples. The required structure for a variety of these systems will appear from the description below. In addition, the techniques are not described with reference to any particular programming language, and various examples may thus be implemented using a variety of programming languages.

In various implementations, the system operates as a standalone device or may be connected (e.g., networked) to other systems. In a networked deployment, the system may operate in the capacity of a server or a client system in a client-server network environment, or as a peer system in a peer-to-peer (or distributed) network environment.

The system may be a server computer, a client computer, a personal computer (PC), a tablet PC (e.g., an iPad®, a Microsoft Surface®, a Chromebook®, etc.), a laptop computer, a set-top box (STB), a personal digital assistants (PDA), a mobile device (e.g., a cellular telephone, an iPhone®, and Android® device, a Blackberry®, etc.), a wearable device, an embedded computer system, an electronic book reader, a processor, a telephone, a web appliance, a network router, switch or bridge, or any system capable of executing a set of instructions (sequential or otherwise) that specify actions to be taken by that system. The system may also be a virtual system such as a virtual version of one of the aforementioned devices that may be hosted on another computer device such as the computer device 902.

In general, the routines executed to implement the implementations of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while examples have been described in the context of fully functioning computers and computer systems, those skilled in the art will appreciate that the various examples are capable of being distributed as a program object in a variety of forms, and that the disclosure applies equally regardless of the particular type of machine or computer-readable media used to actually effect the distribution.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list of all examples in which a change in state for a binary one to a binary zero or vice-versa in a memory device may comprise a transformation, such as a physical transformation. Rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

The above description and drawings are illustrative and are not to be construed as limiting or restricting the subject matter to the precise forms disclosed. Persons skilled in the relevant art can appreciate that many modifications and variations are possible in light of the above disclosure and may be made thereto without departing from the broader scope of the embodiments as set forth herein. Numerous specific details are described to provide a thorough understanding of the disclosure. However, in certain instances, well-known or conventional details are not described in order to avoid obscuring the description.

As used herein, the terms “connected,” “coupled,” or any variant thereof when applying to modules of a system, means any connection or coupling, either direct or indirect, between two or more elements; the coupling of connection between the elements can be physical, logical, or any combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, or any combination of the items in the list.

As used herein, the terms “a” and “an” and “the” and other such singular referents are to be construed to include both the singular and the plural, unless otherwise indicated herein or clearly contradicted by context.

As used herein, the terms “comprising,” “having,” “including,” and “containing” are to be construed as open-ended (e.g., “including” is to be construed as “including, but not limited to”), unless otherwise indicated or clearly contradicted by context.

As used herein, the recitation of ranges of values is intended to serve as a shorthand method of referring individually to each separate value falling within the range, unless otherwise indicated or clearly contradicted by context. Accordingly, each separate value of the range is incorporated into the specification as if it were individually recited herein.

As used herein, use of the terms “set” (e.g., “a set of items”) and “subset” (e.g., “a subset of the set of items”) is to be construed as a nonempty collection including one or more members unless otherwise indicated or clearly contradicted by context. Furthermore, unless otherwise indicated or clearly contradicted by context, the term “subset” of a corresponding set does not necessarily denote a proper subset of the corresponding set but that the subset and the set may include the same elements (i.e., the set and the subset may be the same).

As used herein, use of conjunctive language such as “at least one of A, B, and C” is to be construed as indicating one or more of A, B, and C (e.g., any one of the following nonempty subsets of the set {A, B, C}, namely: {A}, {B}, {C}, {A, B}, {A, C}, {B, C}, or {A, B, C}) unless otherwise indicated or clearly contradicted by context. Accordingly, conjunctive language such as “as least one of A, B, and C” does not imply a requirement for at least one of A, at least one of B, and at least one of C.

As used herein, the use of examples or exemplary language (e.g., “such as” or “as an example”) is intended to more clearly illustrate embodiments and does not impose a limitation on the scope unless otherwise claimed. Such language in the specification should not be construed as indicating any non-claimed element is required for the practice of the embodiments described and claimed in the present disclosure.

As used herein, where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

Those of skill in the art will appreciate that the disclosed subject matter may be embodied in other forms and manners not shown below. It is understood that the use of relational terms, if any, such as first, second, top and bottom, and the like are used solely for distinguishing one entity or action from another, without necessarily requiring or implying any such actual relationship or order between such entities or actions.

While processes or blocks are presented in a given order, alternative implementations may perform routines having steps, or employ systems having blocks, in a different order, and some processes or blocks may be deleted, moved, added, subdivided, substituted, combined, and/or modified to provide alternative or sub combinations. Each of these processes or blocks may be implemented in a variety of different ways. Also, while processes or blocks are at times shown as being performed in series, these processes or blocks may instead be performed in parallel, or may be performed at different times. Further any specific numbers noted herein are only examples: alternative implementations may employ differing values or ranges.

The teachings of the disclosure provided herein can be applied to other systems, not necessarily the system described above. The elements and acts of the various examples described above can be combined to provide further examples.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further examples of the disclosure.

These and other changes can be made to the disclosure in light of the above Detailed Description. While the above description describes certain examples, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific implementations disclosed in the specification, unless the above Detailed Description section explicitly defines such terms. Accordingly, the actual scope of the disclosure encompasses not only the disclosed implementations, but also all equivalent ways of practicing or implementing the disclosure under the claims.

While certain aspects of the disclosure are presented below in certain claim forms, the inventors contemplate the various aspects of the disclosure in any number of claim forms. Any claims intended to be treated under 45 U.S.C. § 112(f) will begin with the words “means for”. Accordingly, the applicant reserves the right to add additional claims after filing the application to pursue such additional claim forms for other aspects of the disclosure.

The terms used in this specification generally have their ordinary meanings in the art, within the context of the disclosure, and in the specific context where each term is used. Certain terms that are used to describe the disclosure are discussed above, or elsewhere in the specification, to provide additional guidance to the practitioner regarding the description of the disclosure. For convenience, certain terms may be highlighted, for example using capitalization, italics, and/or quotation marks. The use of highlighting has no influence on the scope and meaning of a term; the scope and meaning of a term is the same, in the same context, whether or not it is highlighted. It will be appreciated that same element can be described in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various examples given in this specification.

Without intent to further limit the scope of the disclosure, examples of instruments, apparatus, methods and their related results according to the examples of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

Some portions of this description describe examples in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module is implemented with a computer program object comprising a computer-readable medium containing computer program code, which can be executed by a computer processor for performing any or all of the steps, operations, or processes described.

Examples may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Examples may also relate to an object that is produced by a computing process described herein. Such an object may comprise information resulting from a computing process, where the information is stored on a non-transitory, tangible computer readable storage medium and may include any implementation of a computer program object or other data combination described herein.

The language used in the specification has been principally selected for readability and instructional purposes, and it may not have been selected to delineate or circumscribe the subject matter. It is therefore intended that the scope of this disclosure be limited not by this detailed description, but rather by any claims that issue on an application based hereon. Accordingly, the disclosure of the examples is intended to be illustrative, but not limiting, of the scope of the subject matter, which is set forth in the following claims.

Specific details were given in the preceding description to provide a thorough understanding of various implementations of systems and components for a contextual connection system. It will be understood by one of ordinary skill in the art, however, that the implementations described above may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method of generating content using sequences of model prompts to be processed by machine-learning models, the computer-implemented method comprising:
providing a prompt-schema interface that identifies one or more machine-learning models for generating content and a plurality of model prompts to be processed by the one or more machine-learning models;
receiving, via the prompt-schema interface, a selection of a machine-learning model from the one or more machine-learning models and a sequence of model prompts from the plurality of model prompts;
processing the sequence of model prompts using the machine-learning model to generate a set of intermediate responses, wherein processing the sequence includes, for each model prompt of the sequence:
accessing a previously-generated response, wherein the previously-generated response was generated by processing a previous model prompt using the machine-learning model; and
processing the model prompt of the sequence and the previously-generated response using the machine-learning model to generate an intermediate response; and
generating a target content based on one or more intermediate responses of the set of intermediate responses.

2. The computer-implemented method of claim 1, wherein the previously-generated response corresponds to another intermediate response of the set of intermediate responses, and wherein the previous model prompt corresponds to another model prompt of the sequence.

3. The computer-implemented method of claim 1, wherein the sequence of the model prompts is sequentially processed using the machine-learning model.

4. The computer-implemented method of claim 1, wherein processing the model prompt includes:
determining that the model prompt corresponds to a branching prompt;
processing the branching prompt using the machine-learning model to generate a set of processing threads, wherein each processing thread of the set of processing threads is configured to generate a portion of the intermediate response; and
simultaneously processing the set of processing threads in parallel to generate the portions of the intermediate response, wherein generating the target content includes aggregating the portions of the intermediate response.

5. The computer-implemented method of claim 1, wherein the machine-learning model is a large-language model (LLM).

6. The computer-implemented method of claim 1, wherein the prompt-schema interface provides a first set of graphical user-interface elements that represent the one or more machine-learning models, and wherein the machine-learning model is selected based on one or more user interactions with the first set of graphical user-interface elements.

7. The computer-implemented method of claim 1, wherein the prompt-schema interface provides a second set of graphical user-interface elements that represent the sequence of model prompts, and wherein the sequence is selected based on one or more user interactions with the second set of graphical user-interface elements.

8. The computer-implemented method of claim 1, wherein the target content includes a data-collection script, a disease report, and/or a patient-recruitment content associated with a clinical trial.

9. The computer-implemented method of claim 1, wherein the target content includes one or more inclusion or exclusion criteria associated with a clinical trial.

10. The computer-implemented method of claim 1, wherein processing the model prompt includes:
accessing supplemental-input data, wherein the supplemental-input data is associated with one or more model prompts of the sequence of model prompts; and
processing the model prompt, the previously-generated response, and the supplemental-input data using the machine-learning model to generate the intermediate response.

11. A system comprising:
one or more processors; and
memory storing thereon instructions that, as a result of being executed by the one or more processors, cause the system to perform operations comprising:
providing a prompt-schema interface that identifies one or more machine-learning models for generating content and a plurality of model prompts to be processed by the one or more machine-learning models;
receiving, via the prompt-schema interface, a selection of a machine-learning model from the one or more machine-learning models and a sequence of model prompts from the plurality of model prompts;
processing the sequence of model prompts using the machine-learning model to generate a set of intermediate responses, wherein processing the sequence includes, for each model prompt of the sequence:
accessing a previously-generated response, wherein the previously-generated response was generated by processing a previous model prompt using the machine-learning model; and
processing the model prompt of the sequence and the previously-generated response using the machine-learning model to generate an intermediate response; and
generating a target content based on one or more intermediate responses of the set of intermediate responses.

12. The system of claim 11, wherein the previously-generated response corresponds to another intermediate response of the set of intermediate responses, and wherein the previous model prompt corresponds to another model prompt of the sequence.

13. The system of claim 11, wherein the sequence of the model prompts is sequentially processed using the machine-learning model.

14. The system of claim 11, wherein processing the model prompt includes:
determining that the model prompt corresponds to a branching prompt;
processing the branching prompt using the machine-learning model to generate a set of processing threads, wherein each processing thread of the set of processing threads is configured to generate a portion of the intermediate response; and
simultaneously processing the set of processing threads in parallel to generate the portions of the intermediate response, wherein generating the target content includes aggregating the portions of the intermediate response.

15. The system of claim 11, wherein the machine-learning model is a large-language model (LLM).

16. The system of claim 11, wherein the prompt-schema interface provides a first set of graphical user-interface elements that represent the one or more machine-learning models, and wherein the machine-learning model is selected based on one or more user interactions with the first set of graphical user-interface elements.

17. The system of claim 11, wherein the prompt-schema interface provides a second set of graphical user-interface elements that represent the sequence of model prompts, and wherein the sequence is selected based on one or more user interactions with the second set of graphical user-interface elements.

18. The system of claim 11, wherein the target content includes a data-collection script, a disease report, and/or a patient-recruitment content associated with a clinical trial.

19. The system of claim 11, wherein the target content includes one or more inclusion or exclusion criteria associated with a clinical trial.

20. The system of claim 11, wherein processing the model prompt includes:
accessing supplemental-input data, wherein the supplemental-input data is associated with one or more model prompts of the sequence of model prompts; and
processing the model prompt, the previously-generated response, and the supplemental-input data using the machine-learning model to generate the intermediate response.

21. A non-transitory, computer-readable storage medium storing thereon executable instructions that, as a result of being executed by one or more processors of a computer system, cause the computer system to perform operations comprising:
providing a prompt-schema interface that identifies one or more machine-learning models for generating content and a plurality of model prompts to be processed by the one or more machine-learning models;
receiving, via the prompt-schema interface, a selection of a machine-learning model from the one or more machine-learning models and a sequence of model prompts from the plurality of model prompts;
processing the sequence of model prompts using the machine-learning model to generate a set of intermediate responses, wherein processing the sequence includes, for each model prompt of the sequence:
accessing a previously-generated response, wherein the previously-generated response was generated by processing a previous model prompt using the machine-learning model; and
processing the model prompt of the sequence and the previously-generated response using the machine-learning model to generate an intermediate response; and
generating a target content based on one or more intermediate responses of the set of intermediate responses.

22. The non-transitory, computer-readable storage medium of claim 21, wherein the previously-generated response corresponds to another intermediate response of the set of intermediate responses, and wherein the previous model prompt corresponds to another model prompt of the sequence.

23. The non-transitory, computer-readable storage medium of claim 21, wherein the sequence of the model prompts is sequentially processed using the machine-learning model.

24. The non-transitory, computer-readable storage medium of claim 21, wherein processing the model prompt includes:
determining that the model prompt corresponds to a branching prompt;
processing the branching prompt using the machine-learning model to generate a set of processing threads, wherein each processing thread of the set of processing threads is configured to generate a portion of the intermediate response; and
simultaneously processing the set of processing threads in parallel to generate the portions of the intermediate response, wherein generating the target content includes aggregating the portions of the intermediate response.

25. The non-transitory, computer-readable storage medium of claim 21, wherein the machine-learning model is a large-language model (LLM).

26. The non-transitory, computer-readable storage medium of claim 21, wherein the prompt-schema interface provides a first set of graphical user-interface elements that represent the one or more machine-learning models, and wherein the machine-learning model is selected based on one or more user interactions with the first set of graphical user-interface elements.

27. The non-transitory, computer-readable storage medium of claim 21, wherein the prompt-schema interface provides a second set of graphical user-interface elements that represent the sequence of model prompts, and wherein the sequence is selected based on one or more user interactions with the second set of graphical user-interface elements.

28. The non-transitory, computer-readable storage medium of claim 21, wherein the target content includes a data-collection script, a disease report, and/or a patient-recruitment content associated with a clinical trial.

29. The non-transitory, computer-readable storage medium of claim 21, wherein the target content includes one or more inclusion or exclusion criteria associated with a clinical trial.

30. The non-transitory, computer-readable storage medium of claim 21, wherein processing the model prompt includes:
accessing supplemental-input data, wherein the supplemental-input data is associated with one or more model prompts of the sequence of model prompts; and
processing the model prompt, the previously-generated response, and the supplemental-input data using the machine-learning model to generate the intermediate response.
